(19) United States
(12) Patent Application Publication
(10) Pub. No.: US 2005/0148554 A1
(43) Pub. Date: Jul. 7, 2005

(54) Anti-inflammatory and anti-thrombotic compounds and their compositions

(76) Inventors: Don Zhang, Old Lyme, CT (US); Guojian Xie, Cheshire, CT (US); Zhengwei Huan, Cheshire, CT (US); Hang Chen, Waterbury, CT (US); Charles Davis, Meriden, CT (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/986,974

(22) Filed: Nov. 12, 2004

Related U.S. Application Data
(63) Continuation of application No. 10/752,142, filed on Jan. 6, 2004, now abandoned.
(60) Provisional application No. 60/439,714, filed on Jan. 13, 2003.

(51) Int. Cl. A61K 31/60; C07C 23/02

(52) U.S. Cl. 514/159; 558/482

(57) ABSTRACT

The invention encompasses novel compounds of Formula I, which are anti-inflammatory and anti-thrombotic agents. 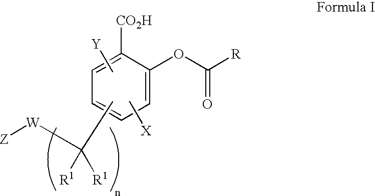 The invention also encompasses certain pharmaceutical compositions and methods for treatment of cyclooxygenases (COX-1 and COX-2) mediated diseases comprising the use of compounds of Formula I. The above compounds may be used as a combination therapy with low-dose aspirin, NSAIDs, or selective COX-2 inhibitors to treat chronic cyclooxygenases mediated diseases or conditions while simultaneously reducing the risk of thrombotic cardiovascular events.

### CROSS-REFERENCE TO RELATED APPLICATIONS

This application is a Continuation of U.S. Serial No 752,142 filed Jan. 6, 2004, which claims the benefit of U.S. Provisional Application Ser. No. 60/439,714 filed Jan. 13, 2003. These applications are incorporated by reference herein in their entireties.

### BACKGROUND OF THE INVENTION

The NSAIDs are the class of drugs known as non-steroidal anti-inflammatory drugs which are active in reducing the prostaglandin-induced pain and swelling associated with the inflammation process but are also active in affecting other prostaglandin-regulated processes not associated with the inflammation process. In particular, Aspirin is one of the most widely used anti-inflammatory and anti-thrombotic agents in the clinics. However, the use of high doses of most common NSAIDs including Aspirin can produce severe side effects, such as life threatening ulcers that limit their therapeutic potential. The gastrointestinal toxicity is inherent to the mechanism of action of NSAIDs, which prevent the production of prostaglandins by inhibiting enzymes in the human arachidonic acid/prostaglandin pathway including the enzyme cyclooxygenases (COX-1 and COX-2).

The commonly known NSAIDs are generally acids and their NO-releasing ester type prodrugs are known in the art and are reported to have improved gastrointestinal and cardiovascular safety profiles over their parent NSAID counterparts. See for example patents WO 94/04484 and WO 94/12463, herein incorporated by reference in their entireties, which describe several groups of compounds including the well-known commercial product Diclofenac, Flurbiprofen, Indoprofen. Particularly noteworthy is WO 97/16405, also incorporated by reference in its entirety, which describes nitrate-containing esters of Aspirin. However, the efficacy of this type of Aspirin ester containing prodrugs is compromised due to the competing deacetylation of the prodrugs by esterases, thus resulting in the loss of the anti-inflammatory and anti-thrombotic activities of Aspirin (see the following scheme).

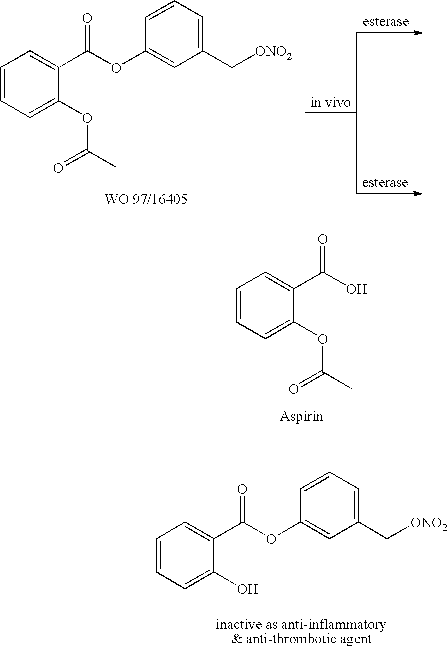

### SUMMARY OF THE INVENTION

The present invention provides a novel class of anti-inflammatory and anti-thrombotic agents, which inhibit cyclooxygenases (COX-1 and COX-2) through the same mechanism as Aspirin and yet have all of the desired properties of nitric ester. The novel nitric esters containing agents useful for treating cyclooxygenases (COX-1 and COX-2) mediated diseases or conditions, which can be administered alone or in combination with NSAIDs or selective cyclooxygenase-2 inhibitors. Thus, the invention provides for a clearly superior profile than that hitherto obtainable in that it provides efficacy in treating chronic cyclooxygenases (COX-1 and COX-2) mediated diseases or conditions, effectively reducing the risk of thrombotic cardiovascular events and renal side effects and at the same time reduces the risk of GI ulceration or bleeding (see the following scheme).

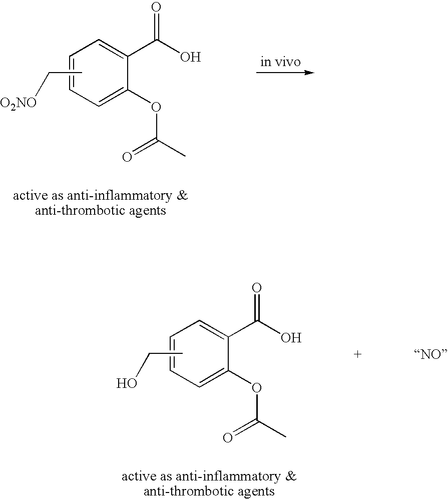

The invention encompasses novel compounds of Formula I, which are nitric oxide-releasing anti-inflammatory, and anti-thrombotic drugs useful in the treatment of cyclooxygenases mediated diseases.

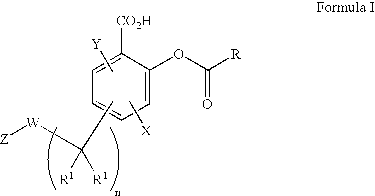

The invention also encompasses certain pharmaceutical compositions and methods for treatment of cyclooxygenases (COX-1 and COX-2) mediated diseases comprising the use of compounds of Formula I. The above compounds may be used as a combination therapy with NSAIDs or selective COX-2 inhibitors to treat chronic cyclooxygenases (COX-1 and COX-2) mediated diseases or conditions while simultaneously reducing the risk of gastrointestinal toxicity and thrombotic cardiovascular events.

### DETAILED DESCRIPTION OF THE INVENTION

The invention encompasses novel compounds of Formula I, which are nitric oxide-releasing anti-inflammatory and anti-thrombotic drugs useful in the treatment of cyclooxygenases mediated diseases, 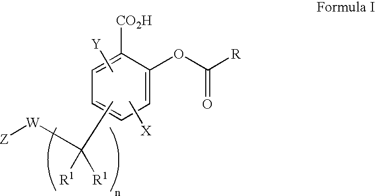
or a pharmaceutically acceptable salt thereof wherein
R is methyl, ethyl, or a linear or branched C₃-C₅ alkyl or the residue of heterocycle with a single ring having 5 or 6 atoms which may be aromatic, partially or totally hydrogenated, containing one or more heteroatoms independently chosen from O, N, S;
X and Y are independently chosen from H, halogen, CF₃, SC₁-C₃ alkyl, OC₁-C₃ alkyl;
n=0-6;
R¹ is C₁₋₆ alkyl, aryl, or the two R¹s can be joined with a bond to form a carbocyclic ring;
W is O, S, NR⁴ wherein R⁴ is H or a linear or branched alkyl having 1 to 10 carbon atoms, preferably 1 to 4 carbon atoms;
Z is H, NO₁₋₂ or Linker-NO₁₋₂ wherein the Linker is selected from the group consisting of:
   (a) —C(O)—(CR²R³)ₙW—, wherein n is 1, 2, 3 or 4, and R² and R³ are selected from H, C₁₋₆ alkyl, C₁₋₆cycloalkyl, aryl or heteroaryl;
   (b) —C(O)—C₃₋₆cycloalkylW—, wherein the C₃₋₆cycloalkyl optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,
   (c) —C(O)—O(CR²R³)ₙW—, wherein n is 1, 2, 3 or 4, and R² and R³ are selected from C₁₋₆ alkyl, C₁₋₆cycloalkyl, aryl or heteroaryl,
   (d) —C(O)—OC₃₋₆cycloalkylW—, wherein the C₃₋₆cycloalkyl optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,
   (e) aryl, wherein the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,
   (f) Heteroaryl optionally mono-, di- or tri-substituted with substituents selected from the group consisting of,
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,
   (h) —C(O)-aryl-(CR²R³)—W—, wherein n=0-6 and the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C α-₃alkyl,
      (11) C₁₋₃alkylthio,
   (i) —C(O)-heteroaryl-(CR²R³)ₙ—W—, wherein n=0-6 and the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,
   (j) —C(O)—O-aryl-(CR²R³)ₙ—W—, wherein n=0-6 and the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (10) CF₃,
      (11) CN;
      (12)CH₂COOH
      (10) CH₂COO—C₁₋₃alkyl,
      (11) C₁₋₃alkylthio,
   (k) —C(O)—O-heteroaryl-(CR²R³)ₙ—W—, wherein n=0-6 and the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkylthio,

For purposes of this specification, heteroaryl or benzoheteroaryl group includes benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

The compounds of the present invention are nitric oxide releasing prodrugs, which liberate nitric oxide and cyclooxygenase inhibitors in vivo and can be administered alone or in combination with NSAIDs or selective COX-2 inhibitors. Thus, the invention provides for a clearly superior profile than that hitherto obtainable in that it provides efficacy in treating chronic cyclooxygenases mediated diseases or conditions, effectively reducing the risk of thrombotic cardiovascular events and renal side effects and at the same time reduces the risk of GI ulceration or bleeding.

The invention also encompasses a pharmaceutical composition comprising a compound of Formula I and a pharmaceutically acceptable carrier.

A “thrombotic cardiovascular event” is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fugax), reversible ischemic neurologic deficits, and any similar thrombotic event in any vascular bed (splanchnic, renal, aortic, peripheral, etc.).

The pharmaceutical compositions of the present invention comprise a compound of Formula I as an active ingredient or a pharmaceutically acceptable salt, thereof, and may also contain a pharmaceutically acceptable carrier and optionally other therapeutic or inert ingredients.

The compounds of the invention are preferably administered internally, e.g., intravenously, in the form of conventional pharmaceutical preparations, for example in conventional enteral or parenteral pharmaceutically acceptable excipients containing organic and/or inorganic inert carriers, such as water, gelatin, lactose, starch, magnesium stearate, talc, plant oils, gums, alcohol, Vaseline, or the like. The pharmaceutical preparations can be in conventional solid forms, for example, tablets, dragees, suppositories, capsules, or the like, or conventional liquid forms, such as suspensions, emulsions, or the like. If desired, they can be sterilized and/or contain conventional pharmaceutical adjuvants, such as preservatives, stabilizing agents, wetting agents, emulsifying agents, buffers, or salts used for the adjustment of osmotic pressure. The pharmaceutical preparations may also contain other therapeutically active materials.

The pharmaceutical preparation of the invention should include an amount of the compound of the invention effective for antiinflammatory and/or antithrombotic activity. The effective dosage will depend on the antiinflammatory and/or antithrombotic activity of the particular compound employed and is thus within the ordinary skill of the art to determine for any particular host mammal or other host organism. Suitable dosages may be, for example, in the range of about 0.5-100 mg per kg for a human being. Alternatively, the claimed compounds may be used to control proliferation of neoplastic cells in vitro or they may be used as antineoplastic agents in nonhuman mammals.

The Compound of Formula I is useful for the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, following surgical and dental procedures. In addition, such a compound may inhibit cellular neoplastic transformations and metastic tumor growth and hence can be used in the treatment of cancer. Compounds of Formula I may also be useful for the treatment of dementia including pre-senile and senile dementia, and in particular, dementia associated with Alzheimer Disease (i.e. Alzheimer's dementia).

Compounds of Formula I will also inhibit prostanoid-induced smooth muscle contraction by preventing the synthesis of contractile prostanoids and hence may be of use in the treatment of dysmenorrhea, premature labor and asthma.

### Methods of Synthesis

### Method A

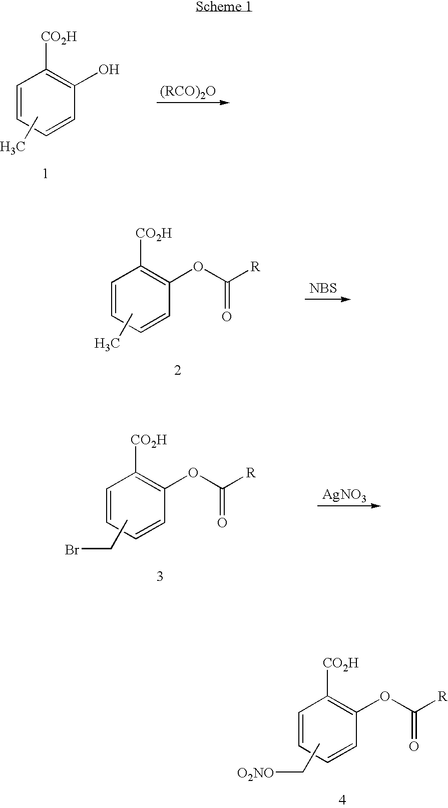

Esterification of a methylsalicylic acid 1 with an acid anhydride, such as acetic anhydride, can provide ester-acid 2. Bromination of 2 with a brominating agent such as N-Bromo Succinimide (NBS) yields bromide 3 which can be converted to the final product 4 upon nitration with silver nitrate (Scheme 1).

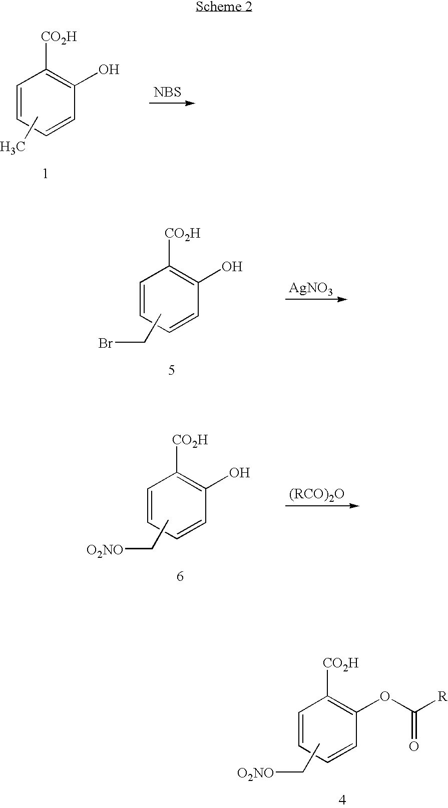

Alternatively, the methylsalicylic acid can be brominated first to give bromide 5, which can then be nitrated with silver nitrate followed by esterification to afford the desired product 4.

### Method B

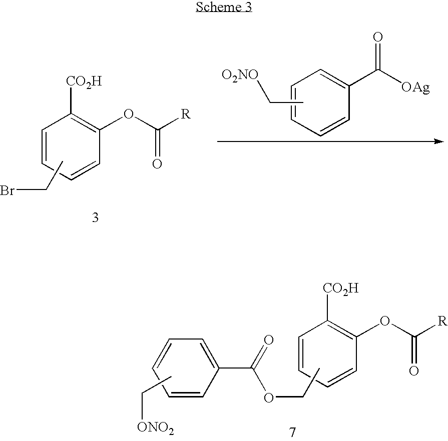

Reaction of bromide 3 with an appropriate nitrate containing benzoic acid silver salt in an inert solvent can provide the desired product of 7.

### Method C

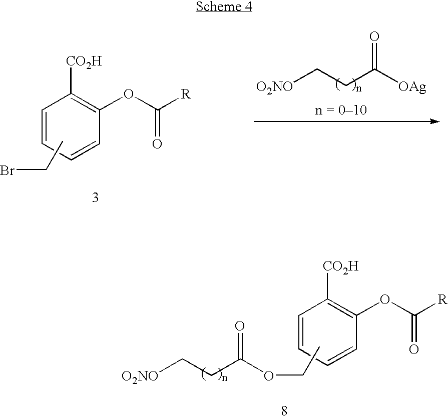

Reaction of bromide 3 with an aliphatic acid silver salt under the same conditions as in Scheme 3 can yields the desired product 8.

### Method D

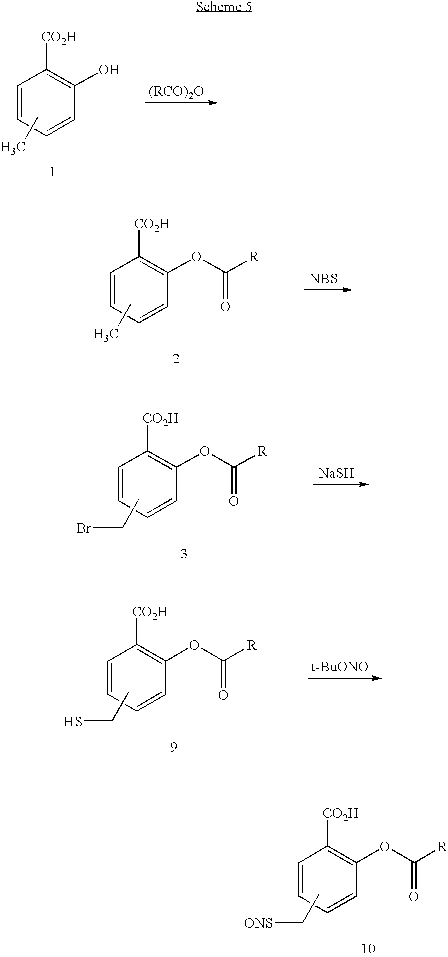

The bromide 3 can be converted to thiol 9, which can then be nitrosylated with t-BuONO to afford the desired product 10.

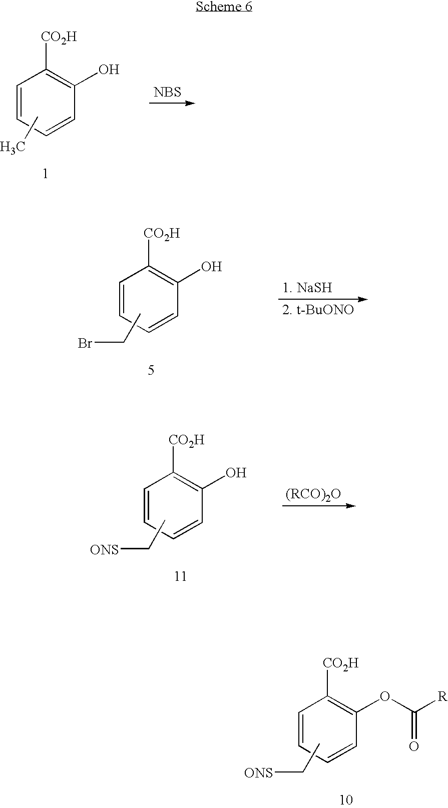

Alternatively, the bromide 5 can be converted to nitrosothiol compound 11, which can then be to the desired compound 10 by esterification.

Assays for Determining Biological Activity

The compound of Formula I can be tested using the following assays to determine their biological activity.

Inhibition of Cyclooxygenase Activity

Compounds are tested as inhibitors of cyclooxygenase activity in whole cell and microsomal cyclooxygenase assays. Both of these assays measure prostaglandin E₂ (PGE₂) synthesis in response to arachidonic acid, using a radioimmunoassay. Cells used for whole cell assays, and from which microsomes are prepared for microsomal assays, are human osteosarcoma 143 cells (which specifically express cyclooxygenase-2) and human U-937 cells (which specifically express cyclooxygenase-1). In these assays, 100% activity is defined as the difference between prostaglandin E₂ synthesis in the absence and presence of arachidonic acid. IC₅₀ values represent the concentration of putative inhibitor required to return PGE₂ synthesis to 50% of that obtained as compared to the uninhibited control.

NSAID-Induced Gastropathy in Rats

Rationale

The major side effect of conventional NSAIDs is their ability to produce gastric lesions in man. Rats are sensitive to the actions of NSAIDs and have been used commonly in the past to evaluate the gastrointestinal side effects of current conventional NSAIDs. In the present assay, NSAID-induced gastrointestinal damage is observed by measuring the seriousness of the gastropathy induced according to the criteria indicated by Wallace et al (Am. J. Physiol. Vol. 259, G642, 1990) herein incorporated by reference in its entirety.

Anti-Aggregating Platelet Assay

The anti platelet aggregation activity is evaluated in vitro on human platelets stimulated by thrombin or arachidonic acid according to the method described by Bertele et al (Science, Vol. 220, P. 517, 1983) herein incorporated by reference in its entirety.

Rat Aortic Smooth Muscle Rings in Male Spargue-Dawley Rats

Preparation of rat aortic smooth muscle rings Male Sprague-Dawley rats (Charles River Laboratories (Wilmington, Mass.) were euthanized by intraperiton injection of a high dose of sodium pentobarbitone (80-100 mg/kg). The thoracic aorta was rapidly excised and immediately placed in a Petri dish containing warm (37° C.) oxygenated (95% 0, and 5% CO₂) Kreb's buffer (composition per millimolar: NaCl (119); KCl (4.69); CaCl₂ 2H₂O (2.52); MgSO₄.7H₂O (0.57); NaHCO₃, (25); NaHPO₄•H₂O (1.01) and glucose (11.1). Under a stereoscopic dissecting microscope, the aorta was cleaned, freed from adhering fat and connective tissues. The tissue was cut into ring segments, each approximately 2-3 mm in length.

For experiments to measure relaxation of the tissue under various conditions, a stainless steel tissue holder and an U-shaped stainless steel wire were inserted into the lumen of the aortic ring. The tissue holder anchored the ring at 142 the bottom of the organ bath whereas the end of the U-shaped steel wire was tied with fine silk thread so that it connected to the FT-202 transducer. The tissue holder and the steel wire along with the aortic ring were then suspended in a 5-ml, double-jacketed temperature-controlled glass organ bath (Radnoti Glass Technology, Inc., Monrovia, Calif.) filled with fresh Kreb's buffer. A mixture of 95% O₂ and 5% CO₂ was bubbled through a porous sintered disc at the bottom of the bath. The rings were given an initial resting tension of 1.5 g and the preparation was allowed to equilibrate at the initial tension for about 90 minutes. During this equilibration period, the bath fluid was changed every 15 minutes and replaced with fresh prewarmed (37° C.) Kreb's buffer. The isometric tension of the aortic muscle at rest and its response to different stimuli were recorded on a Power Macintosh 6100 computer via a MacLab 8/S computer interface (CB Sciences, Inc, Milford, Mass.) after an initial amplification through a low-noise ETH-400 bioamplifier (CB Sciences, Inc, Milford, Mass.). Contractile responsiveness of the tissue strips was established with 10 μM phenylephrine, and the strips were incubated with the drug for 20 minutes to establish a steady level of contraction.

To test the relaxation effects, test compounds were added to the phenylephrine precontracted strips in the tissue bath at cumulative concentrations of 0.1 M to 0.1 mM. Concentration of test compounds was increased only after relaxation at the previous concentration had reached a plateau level.

### REPRESENTATIVE EXAMPLES

The invention will now be illustrated by the following non-limiting examples in which, unless stated otherwise.

All operations were carried out at room or ambient temperature, that is, at a temperature in the range 18-25° C.; evaporation of solvent was carried out using a rotary evaporator under reduced pressure (600-4000 pascals: 4.5-30 mm. Hg) with a bath temperature of up to 60° C.; the course of reactions was followed by thin layer chromatography (TLC) and reaction times are given for illustration only; melting points are uncorrected and ‘d’ indicates decomposition; the melting points given are those obtained for the materials prepared as described; polymorphism may result in isolation of materials with different melting points in some preparations; the structure and purity of all final products were assured by at least one of the following techniques: TLC, mass spectrometry, nuclear magnetic resonance (NMR) spectrometry or microanalytical data; yields are given for illustration only; when given, NMR data is in the form of delta (δ) values for major diagnostic protons, given in parts per million (ppm) relative to tetramethylsilane (TMS) as internal standard, determined at 250 MHz or 400 MHz using the indicated solvent; conventional abbreviations used for signal shape are: s. singlet; d. doublet; t. triplet; m. multiplet; br. broad; etc.: in addition “Ar” signifies an aromatic signal; chemical symbols have their usual meanings; the following abbreviations have also been used v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

The following abbreviations have the indicated meanings:

| | |
|---|---|
| Ac = | Acetyl |
| Bn = | Benzyl |
| DBU = | 1,8-diazabicyclo[5.4.0]undec-7-ene |
| DIBAL = | diisobutylaluminum hydride |
| DMAP = | 4-(dimethylamino)pyridine |
| DMF = | N,N-dimethylformamide |
| Et₃N = | Triethylamine |
| HBSS = | Hanks' balanced salt solution |
| LDA = | lithium diisopropylamide |
| m-CPBA = | Metachloroperbenzoic acid |
| MMPP = | monoperoxyphtalic acid |
| MPPM = | monoperoxyphthalic acid, magnesium salt 6H₂O |
| Ms = | methanesulfonyl = mesyl = S(O)₂Me |
| Ms0 = | methanesulfonate = mesylate |
| NSAID = | non-steroidal anti-inflammatory drug |
| OXONE ® = | 2KHSO₅.KHSO₄.K₂SO₄ |
| PBS = | phosphate buffered saline |
| PCC = | pyridinium chlorochromate |
| PDC = | pyridinium dichromate |
| Ph = | Phenyl |
| Phe = | Benzenediyl |
| Pye = | Pyridinediyl |
| r.t. = | room temperature |
| rac. = | Racemic |
| SAM = | aminosulfonyl or sulfonamide or S(O)₂NH₂ |
| TBAF = | tetra-n-butylammonium fluoride |
| Th = | 2- or 3-thienyl |
| TFAA = | trifluoroacetic acid anhydride |
| THF = | Tetrahydrofuran |
| Thi = | Thiophenediyl |
| TLC = | thin layer chromatography |
| TMS-CN = | trimethylsilyl cyanide |
| Tz = | 1H (or 2H)-tetrazol-5-yl |
| C₃H₅ = | Allyl |

Alkyl Group Abbreviations

| | |
|---|---|
| Me = | Methyl |
| Et = | Ethyl |
| n-Pr = | normal propyl |
| i-Pr = | Isopropyl |
| n-Bu = | normal butyl |
| i-Bu = | Isobutyl |
| s-Bu = | secondary butyl |
| t-Bu = | tertiary butyl |
| c-Pr = | Cyclopropyl |
| c-Bu = | Cyclobutyl |
| c-Pen = | Cyclopentyl |
| c-Hex = | Cyclohexyl |

### Example 1

### 2-Acetoxy-5-Nitrooxymethyl-Benzoic Acid

### Step 1 2-Acetoxy-5-methyl-benzoic Acid

To a 500 mL 3-neck round-bottom flask equipped with a condenser, 3-methylsalicylic acid (8.77 g, 57.60 mmol), Acetic anhydride (16.30 mL, 172.90 mmol), and concentrated Phosphoric acid (0.2 mL) were added. The suspension of the reaction mixture was heated to reflux under nitrogen atmosphere with stirring. After the reaction suspension became a clear colourless solution, the solution was refluxed for another 2 h under nitrogen atmosphere. The reaction solution was then cooled to room temperature and allowed to stay at room temperature overnight. A white precipitation was formed and collected by filtration. The solid was washed with cold water and dried under vacuum overnight to give 10.5 g of the titled compound as a white solid. ¹H NMR (CDCl₃, 300 MHz): δ 7.98 (d, 1H, J=1.2 Hz), 7.96 (d, 1H, J=1.2 Hz), 7.49 (m, 1H), 2.39 (s, 3H), 2.26 (s, 3H).

### Step 2 2-Acetoxy-5-bromomethyl-benzoic Acid

To a 250 mL 3-neck round bottom flask equipped with a condenser, 2-acetoxy-5-methyl-benzoic acid (1.20 g, 6.18 mmol), and carbon tetrachloride (50 mL) were added under nitrogen atmosphere. While the reaction suspension was heated up to reflux, AIBN (5 mg, 0.01 mmol) was added followed by addition of N-bromosuccinimide (1.15 g, 6.49 mmol) by portions over a period of 2 h. The reaction mixture was refluxed for approximately 18 h and TLC (20% ethyl acetate in hexane) indicated complete consumption of the starting material. The solid was filtrated off and the filtrate was then concentrated under reduced pressure to obtain a white solid mixture. The crude product was purified by silica gel chromatography to yield 280 mg of the titled compound as a white solid. ¹H NMR (CDCl₃, 300 MHz): δ 7.97 (d, 1H, J=1.2 Hz), 7.94 (d, 1H, J=1.2 Hz), 7.38 (m, 1H), 4.56 (s, 2H), 2.33 (s, 3H).

### Step 3 2-Acetoxy-5-nitrooxymethyl-benzoic Acid

To a solution of 2-acetoxy-5-bromomethyl-benzoic acid. (68 mg, 0.26 mmol) in acetonitrile (10 mL) in a 100 mL round bottom flask, silver nitrate (88.3 mg, 0.52 mmol) was added at room temperature. A yellowish precipitation was formed instantly. The reaction mixture was stirred overnight and another portion of silver nitrate (44 mg, 0.26 mmol) was added and stirring was continued for another day. The solid was filtrated off and the filtrate was concentrated under reduced pressure and the residue was purified by silica gel chromatography eluted with 20% methanol in methylene chloride to yield 45 mg of the desired product as a pale yellowish solid. ¹H NMR (CDC₃, 300 MHz): δ 8.0 (d, 1H, J=1.2 Hz), 7.98 (d, 1H, J=1.2 Hz), 7.49 (m, 1H), 5.48 (s, 2H), 2.26 (s, 3H).

### Example 2

### 2-Acetoxy-4-Nitrooxymethyl-Benzoic Acid

### Step 1 2-Acetoxy-4-methyl-benzoic Acid

To a 500 mL 3-neck round-bottom flask equipped with a condenser, 4-methylsalicylic acid (6.61 g, 43.4 mmol), acetic anhydride (12.32 mL, 130 mmol), and concentrated Phosphoric acid (0.2 mL) were added. The reaction suspension was heated to reflux under nitrogen atmosphere with stirring. After the reaction suspension became a clear colourless solution, the solution was heated for another 2 h under nitrogen atmosphere. The reaction solution was cooled to room temperature and allowed to stay at room temperature overnight and a white precipitate was formed. The solid was collected by filtration and washed with cold water and dried in vacuum oven at 50° C. overnight to give 6.8 g of the titled compound as a white solid. ¹H NMR (CDCl₃, 300 MHz): δ 8.0 (d, 1H, J=8.0 Hz), 7.15 (d, 1H, J=8.0 Hz), 6.96 (s, 1H), 2.43 (s, 3H), 2.35 (s, 3H).

### Step 2 2-Acetoxy-4-bromomethyl-benzoic Acid

From 2-acetoxy-4-methyl-benzoic acid, the titled compound was prepared under the same reaction conditions as described in Step 2 of Example 1. ¹H NMR (CDCl₃, 300 MHz): δ 8.10 (d, 1H, J=8.0 Hz), 6.62-7.15 (m, 1H), 6.62 (s, 1H), 4.54 (s, 2H), 2.41 (s, 3H).

### Step 3 2-Acetoxy-4-nitrooxymethyl-benzoic Acid

From 4-bromomethylacetylsalisilic acid, the titled compound was prepared under the same reaction conditions as described in Step 3 of Example 1. ¹H NMR (CD₃Cl, 300 MHz): δ 8.17 (d, 1H, J=8.0 Hz), 7.27-7.40 (m, 1H), 7.19 (m, 1H), 5.49 (s, 2H), 2.37 (s, 3H).

While the invention has been described in combination with embodiments thereof, it is evident that many alternatives, modifications and variations will be apparent to those skilled in the art in light of the foregoing description. Accordingly, it is intended to embrace all such alternatives, modifications and variations as fall within the spirit and broad scope of the appended claims. All patents, patent applications, and publications are hereby incorporated by reference in their entireties.

1. A compound of Formula I: 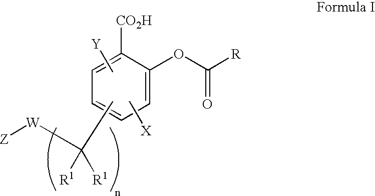
or a pharmaceutically acceptable salt thereof wherein
R is methyl, ethyl, or a linear or branched C₃-C₅ alkyl or the residue of heterocycle with a single ring having 5 or 6 atoms which may be aromatic, partially or totally hydrogenated, containing one or more heteroatoms independently chosen from O, N, S;
X and Y are independently chosen from H, halogen, CF₃, SC₁₋C₃ alkyl, OC₁₋C₃ alkyl;
n=0-6;
W is O, S NH, NR¹ wherein R¹ is a linear or branched alkyl having 1 to 10 carbon atoms, preferably 1 to 4 carbon atoms;
Z is H, NO₁₋₂ or Linker-NO₁₋₂ wherein the Linker is selected from the group consisting of:
(a) —C(O)—(CR²R³)ₙW, wherein n is 0, 1, 2, 3 or 4, and R² and R³ are selected from H, C₁₋₆ alkyl, C₁₋₆cycloalkyl, aryl or heteroaryl;
(b) —C(O)—C₃₋₆cycloalkylW—, wherein the C₃₋₆cycloalkyl optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(c) —C(O)—O(CR²R³)ₙW—, wherein n is 0, 1, 2, 3 or 4, and R² and R³ are selected from C₁₋₆ alkyl, C₁₋₆cycloalkyl, aryl or heteroaryl,
(d) —C(O)—OC₃₋₆cycloalkylW—, wherein the C₃₋₆cycloalkyl optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(e) aryl, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(f) Heteroaryl optionally mono-, di- or tri-substituted with substituents selected from the group consisting of,
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(g) —C(O)-aryl-(CR²R³)ₙ—W—, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(h) —C(O)-heteroaryl-(CR²R³)—W—, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(i) —C(O)—O-aryl-(CR²R³)ₙ—W—, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(j) —C(O)—O-heteroaryl-(CR²R³)ₙ—W—, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,

2. Compounds or their pharmaceutical compositions according to claim 1, having the general formula: 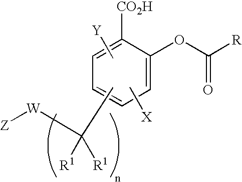
or a pharmaceutically acceptable salt thereof wherein
n is 0, 1, 2, 3 or 4.

3. Cmpounds or their pharmaceutical compositions according to claim 1, having the general formula: 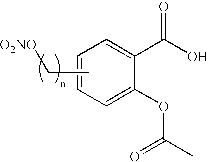
or a pharmaceutically acceptable salt thereof wherein
n is 0, 1, 2, 3 or 4.

4. Compounds or their pharmaceutical compositions according to claim 1, having the general formula: 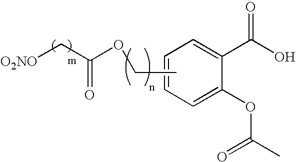
or a pharmaceutically acceptable salt thereof wherein
n and m are independently 0, 1, 2, 3 or 4.

5. Compounds or their pharmaceutical compositions according to claim 1, having the general formula: 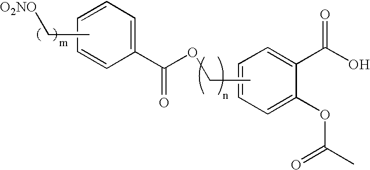
or a pharmaceutically acceptable salt thereof wherein
n and m are independently 0, 1, 2, 3 or 4.

6. Compounds or their pharmaceutical compositions according to claim 1, having the general formula: 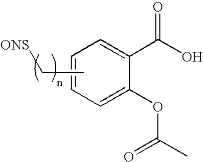
or a pharmaceutically acceptable salt thereof wherein
n is 0, 1, 2, 3 or 4.

7. Compounds or their pharmaceutical compositions according to claim 1, having the general formula: 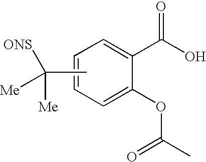
or a pharmaceutically acceptable salt thereof.

8. A method of treating an inflammatory disease susceptible to treatment with a non-steroidal anti-inflammatory agent comprising administering to a patient in need of such treatment of a non-toxic therapeutically effective amount of a compound or pharmaceutical composition according to claim 1.

9. A method of treating cyclooxygenase mediated diseases advantageously treated by combination of a compound according to claim 1 with a selective COX-2 inhibitor.

10. The method according to claim 8 wherein the compound according to claim 1 is administered orally on a once daily, twice daily or three time daily basis.

11. The method according to claim 7 wherein aspirin is administered at a dose of about 30 mg to about 1 g.

12. A method of treating cyclooxygenases mediated diseases advantageously treated by combination of a compound according to claim 1 with NSAIDs.

13. A pharmaceutical composition comprising a compound according to claim 1 and NSAIDs in combination with a pharmaceutically acceptable carrier.
